(19) United States
(12) Patent Application Publication
(10) Pub. No.: US 2024/0212135 A1
(43) Pub. Date: Jun. 27, 2024

(54) DYNAMICALLY ALTERING TRANSPARENCY LEVEL IN SUB-VOLUMES OF ANATOMICAL MAPS

(71) Applicant: BIOSENSE WEBSTER (ISRAEL) LTD., Yokneam (IL)

(72) Inventors: Assaf Govari, Haifa (IL); Andres Claudio Altmann, Haifa (IL)

(21) Appl. No.: 18/086,849

(22) Filed: Dec. 22, 2022

(51) Int. Cl. G06T 7/00; A61B 18/14; G06T 5/00

(52) U.S. Cl. CPC G06T 7/0012; A61B 18/1492; G06T 5/007; A61B 2018/00577; G06T 2207/30048

(57) ABSTRACT

A method including receiving, in an anatomical map of an organ, a position of a tag formed in the organ responsively to a medical procedure carried out at the position. A transparency level is dynamically altered in a sub-volume of the anatomical map of the organ surrounding the tag. The anatomical map is displayed using a first transparency level, and the sub-volume surrounding the tag is displayed using a second transparency level, different from the first transparency level.

### FIELD OF THE DISCLOSURE

The present disclosure relates generally to medical devices, and particularly to methods and systems for improving the presentation and visualization of features in anatomical maps.

### BACKGROUND OF THE DISCLOSURE

Various techniques for presenting features over anatomical maps have been published. One of the challenges is to visualize the features of interest while retaining a clear presentation of the three-dimensional structure of the organ. This combination is important to provide a user (e.g., a physician) with a clear visualization of features of interest within the inner volume of the organ in question, while presenting the general structure of the organ.

The present disclosure will be more fully understood from the following detailed description of the examples thereof, taken together with the drawings in which:

### BRIEF DESCRIPTION OF THE DRAWINGS

FIG. 1 is a schematic, pictorial illustration of a catheter-based electrophysiology mapping and ablation system, in accordance with an example of the present disclosure;

FIGS. 2A, 2B, 2C and 2D are schematic, pictorial illustrations of electro-anatomical (EA) maps of heart tissue, in accordance with examples of the present disclosure;

FIG. 3 is a flow chart that schematically illustrates a method for dynamically adjusting transparency of sub-volumes in an EA map of the heart, in accordance with an example of the present disclosure; and

FIG. 4 is a flow chart that schematically illustrates a method for dynamically adjusting transparency of sub-volumes having ablation tags in an EA map of the heart, in accordance with another example of the present disclosure.

### DETAILED DESCRIPTION OF EXAMPLES

### Overview

Electro-anatomical (EA) mapping of an organ, such as a heart, may comprise (i) moving a distal tip of a catheter within the volume of the heart, (ii) acquiring electrophysiological (EP) signals on inner and outer surfaces of the heart, and (iii) displaying, over the inner and outer surfaces of a three-dimensional (3D) EA map, tags indicative of the acquired signals.

Examples of the present disclosure that are described below, provide techniques for improving the visualization of tags over a 3D EA map of an organ, such as the patient heart, by dynamically altering the transparency of selected portions of the EA map. In some examples, it is possible to display the EA map using a transparent view, in which the surface of the heart is displayed as a transparent tissue, SO as to enable the visualization of tags associated with the inner surfaces of the heart. When moving to the transparent view, the 3D effect of the EA map (e.g., the depth and 3D perception) is lost. The loss of the 3D effect results in a difficulty for a user (e.g., a physician) to understand the topography of the EA map.

In some examples, a system for visualizing the tags without losing the 3D effect of an EA map comprises a processor and a display device, also referred to herein as a display, for brevity. The processor is configured to receive (e.g., from a mapping catheter having sensing electrodes) a first position of a tag that is located within an inner volume of the anatomical map of an organ, and is indicative of an attribute of the organ at the first position. In the present example, the organ comprises a heart and the attribute are based on electrocardiogram (ECG) signals acquired by the electrodes of the catheter.

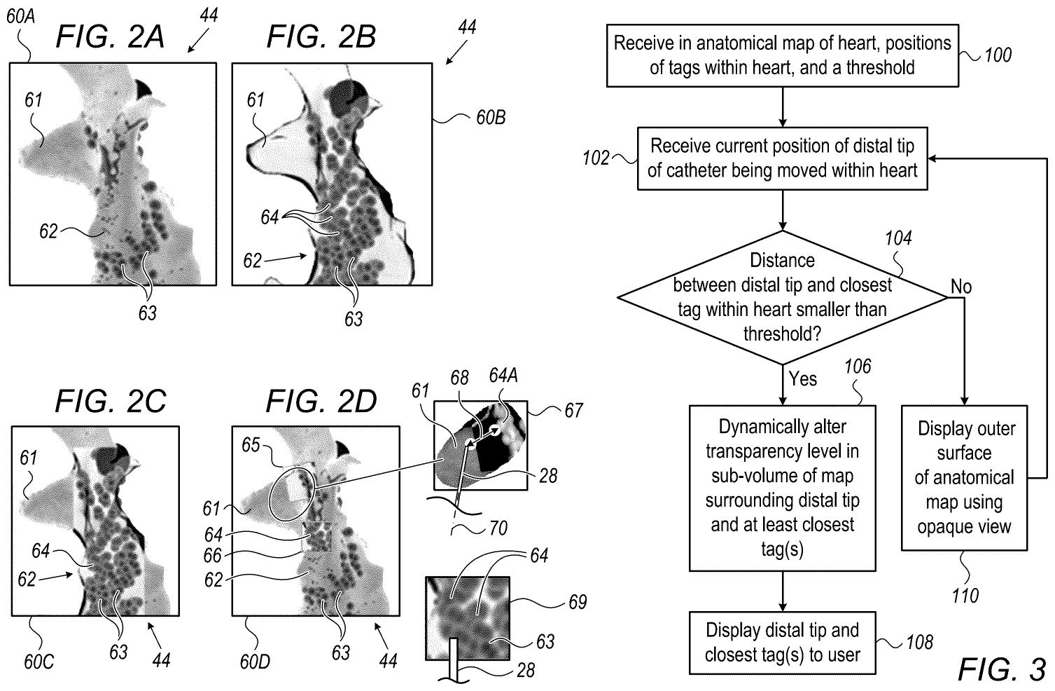

In some examples, the catheter comprises a position sensor configured to generate position signals indicative of the position of a distal tip of the catheter. The processor is further configured to receive one or more second positions, which are the positions of the catheter being moved within the heart. In some examples, the processor is configured to hold at least a threshold, and when the distance between the first position and at least one of the second positions is smaller than the threshold, the processor is configured to alter the transparency level of a sub-volume of the anatomical map that contains the distal tip and the tag. Example implementations of these techniques are described in detail in FIGS. 2C and 2D below.

In some examples, the display is configured to display the sub-volume and the anatomical map to a user (e.g., a physician). In such examples, the processor is configured to retain the 3D effect of the EA map, and yet, to provide the user with the local information about the tag, which is in close proximity with the distal tip. It is noted that based on the disclosed techniques, the transparency level of the sub-volume is altered only when both the distal tip and the tag are located within the same sub-volume. For example, when no tags are located in close proximity to the distal tip, the processor is not altering the transparency level of the EA map, so as to retain the 3D effect thereof.

Additionally, or alternatively, the processor may receive the EA map having a first transparency level, and the tracked position of the distal tip. The processor is configured to dynamically alter the transparency level in a sub-volume of the EA map surrounding the distal tip responsively to the tracked position. In such examples, the display is configured to display (i) the EA map using the first transparency level, and (ii) the sub-volume surrounding the distal tip using a second transparency level, different from the first transparency level. For example, the transparency level of the EA map may be opaque (e.g., showing only the outer surface of the heart), and the only the sub-volume having the distal tip is transparent, so that the user may see the inner volume of the heart (including the inner anatomy, and optionally, the tags) surrounding the distal tip. In other words, the EA map is opaque in order to retain the 3D effect thereof, and only the sub-volumes visited by the distal tip are transparent. Note that the altered transparency is dynamic because the transparency of the sub-volumes is altered responsively to the movement of the distal tip. Example implementations of these techniques are described in detail in FIGS. 2C and 2D below.

The disclosed techniques customize the dynamic visualization of features of interest in EA maps, and more specifically in anatomical maps of organs undergoing a minimal invasive procedure.

### System Description

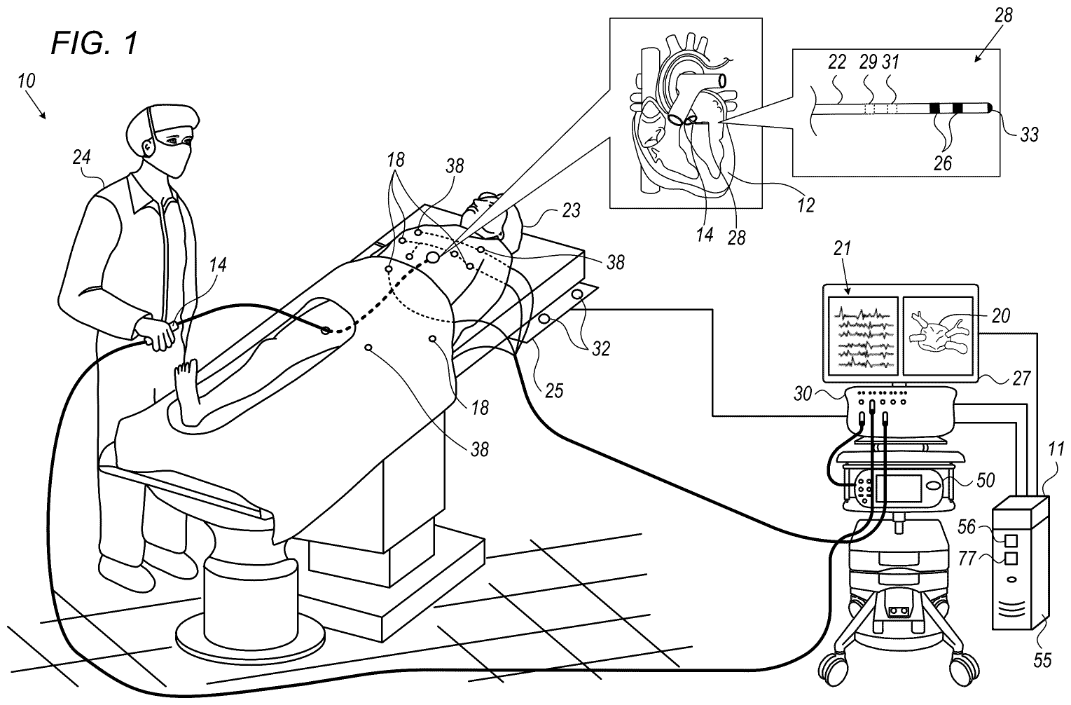

FIG. 1 is a schematic, pictorial illustration of a catheter-based electrophysiology mapping and ablation system 10, in accordance with an example of the present disclosure.

In some examples, system 10 includes multiple catheters, which are percutaneously inserted by a physician 24 through the patient's vascular system into a chamber or vascular structure of a heart 12. Typically, a delivery sheath catheter is inserted into the left or right atrium near a desired location in heart 12. Thereafter, one or more catheters can be inserted into the delivery sheath catheter so as to arrive at the desired location within heart 12. The plurality of catheters may include catheters dedicated for sensing Intracardiac Electrogram (IEGM) signals, catheters dedicated for ablating and/or catheters adapted to carry out both sensing and ablating. An example catheter 14 that is configured for sensing IEGM is illustrated herein. In some embodiments, physician 24 may place a distal tip 28 of catheter 14 in close proximity with or in contact with the heart wall for sensing a target site in heart 12. Additionally, or alternatively, for ablation, physician 24 would similarly place a distal end of an ablation catheter in contact with a target site for ablating tissue intended to be ablated.

In the present example, catheter 14 includes one and preferably multiple electrodes 26 optionally distributed along a shaft 22 at distal tip 28 of catheter 14. Electrodes 26 and configured to sense the IEGM signals. Catheter 14 may additionally include a position sensor 29 embedded in or near distal tip 28 for tracking position and orientation of distal tip 28. Optionally and preferably, position sensor 29 is a magnetic based position sensor including three magnetic coils for sensing three-dimensional (3D) position and orientation.

In some examples, magnetic based position sensor 29 may be operated together with a location pad 25 including a plurality of (e.g., three) magnetic coils 32 configured to generate a plurality of (e.g., three) magnetic fields in a predefined working volume. Real time position of distal tip 28 of catheter 14 may be tracked based on magnetic fields generated with location pad 25 and sensed by magnetic based position sensor 29. Details of the magnetic based position sensing technology are described, for example, in U.S. Pat. Nos. 5,391,199; 5,443,489; 5,558,091; 6,172,499; 6,239,724; 6,332,089; 6,484,118; 6,618,612; 6,690, 963; 6,788, 967; 6,892,091.

In some examples, catheter 14 includes a contact force sensor 31, which is configured to sense the contact force applied to tissue of heart 12 by distal tip 28, and to produce a signal indicative of the sensed contact force.

In some examples, system 10 includes one or more electrode patches 38 positioned for skin contact on patient 23 to establish location reference for location pad 25 as well as impedance-based tracking of electrodes 26. For impedance-based tracking, electrical current is directed toward electrodes 26 and sensed at electrode skin patches 38 so that the location of each electrode can be triangulated via the electrode patches 38. This technique is also referred to herein as Advanced Current Location (ACL) and details of the impedance-based location tracking technology are described in U.S. Pat. Nos. 7,536,218; 7,756,576; 7,848,787; 7,869,865; and 8,456,182. In some embodiments, the magnetic based position sensing and the ACL may be applied concurrently, e.g., for improving the position sensing of one or more electrodes coupled to a shaft of a rigid catheter or to flexible arms or splines at the distal tip of another sort of catheter, such as the PentaRay® or OPTRELL® catheters, available from Biosense Webster, Inc., 31A Technology Drive, Irvine, CA 92618.

In some examples, a recorder 11 displays electrograms 21 captured with body surface ECG electrodes 18 and intracardiac electrograms (IEGM) captured with electrodes 26 of catheter 14. Recorder 11 may include pacing capability for pacing the heart rhythm and/or may be electrically connected to a standalone pacer.

In some examples, system 10 may include an ablation energy generator 50 that is adapted to conduct ablative energy to one or more of electrodes at a distal tip of a catheter configured for ablating. Energy produced by ablation energy generator 50 may include, but is not limited to, radiofrequency (RF) energy or pulse trains of pulsed-field ablation (PFA) energy, including monopolar or bipolar high-voltage DC pulses as may be used to effect irreversible electroporation (IRE), or combinations thereof. In the present example, catheter 14 includes an ablation electrode 33 but optionally includes multiple electrodes 33 (not shown), positioned at distal tip 28 and configured to apply the RF energy and/or the pulse trains of PFA energy to tissue of the wall of heart 12.

In some examples, patient interface unit (PIU) 30 is an interface configured to establish electrical communication between catheters, electrophysiological equipment, power supply and a workstation 55 for controlling the operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

In some examples, workstation 55 includes a storage device, a processor 77 with suitable random-access memory, or storage with appropriate operating software stored therein, an interface 56 configured to exchange signals of data (e.g., between processor 77 and another entity of system 10) and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of multiple catheters within the heart chamber, and (4) displaying on display device 27 sites of interest such as places where ablation energy has been applied or intended to be applied. In some examples, processor 77 is configured to receive position signals from at least one of position sensor 29 and the ACL. Based on the position signals, processor 77 is configured to track the position of distal tip 28, and to display the position of distal tip 28 over map 20. One commercial product embodying elements of the system 10 is available as the CARTO™ 3 System, available from Biosense Webster, Inc., 31A Technology Drive, Irvine, CA 92618.

In some examples, processor 77 receives from contact force sensor 31 a signal indicative of the contact force applied between ablation electrode 33 and the tissue intended to be ablated. Moreover, processor 77 may store one or more contact force thresholds in order to provide physician 24 with an indication of whether the contact force applied between ablation electrode 33 and the tissue intended to be ablated is sufficient for the planned ablation mode (e.g., a first threshold for RF-based ablation mode, and a second different threshold for PFA-based ablation mode).

FIG. 2A is a schematic, pictorial illustration of an electro-anatomical (EA) map 60A of tissue 44 of heart 12, in accordance with an example of the present disclosure. EA map 60A, also referred to herein as map 60A, for brevity, may replace, for example, at least part of map 20 of FIG. 1 above.

In some examples, map 60A comprises multiple sub-volumes (SVs), such as SVs 61 and 62 of heart 12. In the present example, the outer surface of tissue 44 is displayed in map 60A, such that the outer surface appears opaque and the inner volume of heart 12 is not displayed. During the EA mapping of heart 12, physician 24 positions distal tip 28 at selected locations on the surface of tissue 44, and processor 77 receives: (i) the IEGM and/or ECG signals from electrodes 26, and (ii) position signals indicative of the position and orientation of distal tip 28. Based on the IEGM/ECG signals and the position signals, processor 77 is configured to display, over the outer surface of tissue 44 of map 60A, tags 63 indicative of an attribute of heart 12. For example, tags 63 may be indicative of the activation sequences (e.g., local activation times) that may illustrate, for example, the velocity of an EP wave propagating along surface 44. It is noted that electrodes 26 may sense IEGM and/or ECG signals also at the inner surfaces of tissue 44 (and other tissues of heart 12), but because the outer surface of tissue 44 appears opaque, the tags at the inner volume of heart 12 are not displayed on map 60A. Yet, the opaqueness of the outer surface of tissue 44, provides physician 24 with a 3D effect of tissue 44 of heart 12, which may be useful for sensing additional signal at selected positions on the surface of tissue 44 and/or for applying ablating energy to selecting positions on the surface of tissue 44.

FIG. 2B is a schematic, pictorial illustration of EA map 60B of tissue 44 of heart 12, in accordance with an example of the present disclosure. EA map 60B, also referred to herein as map 60B, may replace, for example, at least part of map 20 of FIG. 1 above.

In some examples, processor 77 is configured to hide the outer surface of tissue 44 at SVs 61 and 62, in other words, the outer surface is transparent. Thus, in addition to tags 63 (that are based on signals acquired on the outer surface of tissue 44), processor 77 is configured to display tags 64. In the example of FIGS. 2A and 2B (and also of FIGS. 2C and 2D below), tags 64 appear to be located within the inner volume of EA map 60B. In fact, tags 64 are typically produced when electrodes 26 are positioned against the surface of the inner wall of heart 12, and therefore, tags 64 should be located in the EA map, on the surface of the inner wall of heart 12, rather than within the inner volume of the EA map. This presentation inaccuracy may occur while producing the EA map. Such inaccuracies may happen due to movements that occur, for example, due to the respiration of patient 23, and also in case physician 24 over-presses distal tip 28 against the inner wall of heart 12 while mapping. These phenomena are also referred to as tenting, whose properties and detection techniques are described in detail, for example, in U.S. Pat. No. 8,523,787 to Ludwin et. al, and in U.S. Patent Application Publication 2022/0225925 to Cohen et. al.

Note that in this presentation, physician 24 can see both tags 63 and 64, but the 3D effect of heart 44 is lost in the presentation of map 60B.

FIG. 2C is a schematic, pictorial illustration of EA map 60C of tissue 44 of heart 12, in accordance with an example of the present disclosure. EA map 60C, also referred to herein as map 60C, may replace, for example, at least part of map 20 of FIG. 1 above.

In the example of FIG. 2C, physician 24 moved distal tip 24 along the inner and outer surfaces of SV 62, and did not visit SV 61. In some examples, based on the locations of the signals acquired by electrodes 26, processor 77 is configured to display the outer surface of tissue 44 (i) opaque at SV 61, and (ii) transparent at SV 62. In such examples, processor 77 is configured to: (i) display to physician 24 both tags 63 and 64 located at the outer surface and inner volume of heart 12, and yet, (ii) retain some of the 3D effect of map 60C by displaying the outer surface of tissue 44 opaque at areas (such as SV 61) not visited and/or not having any sort of tags.

FIG. 2D is a schematic, pictorial illustration of EA map 60D of tissue 44 of heart 12, in accordance with an example of the present disclosure. EA map 60D, also referred to herein as map 60D, may replace, for example, at least part of map 20 of FIG. 1 above.

In some examples, processor 77 is configured to dynamically alter a transparency level in one or more sub-volumes of EA map 60D surrounding distal tip 28 responsively to the tracked position of distal tip 28. In the context of the present disclosure and in the claims, the term “dynamically” refers to altering the transparency level in a selected sub-volume of the EA maps (e.g., any of EA maps 20, and 60A-60D) within less than about 2 seconds from the latest movement of distal tip 28. In the example of FIG. 2D, when distal tip 28 is positioned at sub-volumes (SVs) 65 and 66, processor 77 is configured to alter the transparency level of SVs 65 and 66, so as to present both tags 63 and 64 located within SVs 65 and 66.

In the context of the present disclosure and in the claims, the terms “about” or “approximately” for any numerical values or ranges indicate a suitable dimensional tolerance that allows the part or collection of components to function for its intended purpose as described herein.

It is noted that distal tip 28 cannot be at both SVs 65 and 66 at the same time. The embodiments of FIG. 2D, in which distal tip 28 is presented at the same time at both SVs 65 and 66, cannot be carried out using a single distal tip 28, and are presented solely for the sake of describing two different (but related) implementations of the techniques of the present disclosure.

Reference is now made to an inset 69 showing distal tip 28 located within SV 66 of EA map 60D. In the example of inset 69, processor 77 is configured to alter the transparency level of the surface of tissue 44 at SV 66, so as to display tags 63 and 64, and optionally distal tip 28, within SV 66. In this example, based on the positions of tags 63 and 64, and responsively to the position of distal tip 28, processor 77 is configured to dynamically alter the transparency of EA map 60D at SV 66. For example, processor 77 is configured to display the inner volume of SV 66 at a selected distance between about 1 mm and 15 mm from distal tip 28.

In other examples, processor 77 is configured to display the inner volume of heart 12 (e.g., using a transparent view) at a predefined sub-volume surrounding distal tip 28, regardless of the existence of tags or other sort of annotations at the displayed inner volume.

In alternative examples, after applying ablation energy to the tissue of heart 12 at a new position, processor 77 is configured to receive the new position, and/or to generate a new tag (e.g., a new tag 64) at the new position. In such examples, processor 77 is configured to alter the transparency of a new sub-volume in the anatomical map (e.g., of map 60C or 60D) that is surrounding the new tag 64. It is noted that the transparency altering is independent of the position of distal tip 28. For example, in case the new tag 64 is generated at SV 66 while distal tip 28 is positioned at SV 65, processor 77 is configured to alter the transparency of SV 66 even though distal tip 28 is not positioned within or in close proximity to SV 66.

In other examples, instead of using the predefined sub-volume, processor 77 is configured to alter the size of the sub-volume surrounding distal tip 28, e.g., so as to include or exclude tags 63 and 64 and other features of interest located in the surrounding of distal tip 28. For example, (i) in case no tags 64 are located within a sub-volume of about 20 mm surrounding distal tip 28, processor 77 may determine the size of the sub-volume surrounding distal tip 28, to be about 3 mm from distal tip 28, and (ii) in case tags 64 are located within a sub-volume of about 5 mm surrounding distal tip 28, processor 77 may determine the size of the sub-volume surrounding distal tip 28, to be about 10 mm or even 15 mm from distal tip 28.

Note that even though SV 62 contains tags 64 (as shown in FIGS. 2B and 2C), when distal tip 28 is not visiting SV 62, tags 64 thereof are of less interest in physician 24. Thus, processor 77 is configured to visualize to a user (e.g., physician 24) the outer surface of SV 62 opaque, so as to retain the effect of the 3D topography of heart 12 at SV 62.

Reference is now made to an inset 67 showing distal tip 28 located in close proximity with SV 65 of EA map 60D. In the example of inset 67, processor 77 is configured to alter the transparency level of the surface of tissue 44 at SV 65, so as to display tags 63 and 64 (within SV 65), and distal tip 28 located in close proximity thereto. More specifically, processor 77 is configured to hold a threshold for a distance 68 between distal tip 28 and the closest tag 64, in the present example, a tag 64a. Based on the stored positions of tags 64 in EA map 60D, the aforementioned threshold, and responsively to the position of distal tip 28, processor 77 is configured (i) identify when distance 68 (between distal tip 28 and tag 64a) is smaller than the threshold, and (ii) dynamically alter the transparency of EA map 60D at SV 65, in order to visualize to physician 24 (and other users) the inner volume of heart 12 at SV 65.

In some examples, based on the position signals that are indicative of the tracked position of distal tip 28, processor 77 is configured to estimate a movement direction of distal tip 28. For example, based on the orientation of distal tip 28, processor 77 may estimate the movement direction to be approximately parallel to a longitudinal axis 70 of distal tip 28. In some examples, processor 77 may define a first threshold along the movement direction, and a second different threshold along another direction different from the movement direction. In this example, processor 77 may set the first threshold to be about 10 mm, so that any of tags 63 and 64 located less than 10 mm from distal tip 28 along the movement direction is presented to physician 24, e.g., using a transparent view. Moreover, in case one or more tags 64 are positioned, for example, at an angle larger than about 100° relative to the movement direction (referred to herein as behind distal tip 28), processor 77 may set the second threshold to be about 5 mm or even less, because in the next movement of distal tip 28, the distance will increase between distal tip 28 and the tags located there behind. In the example of inset 67, tag 64a is located at a sharp angle relative to longitudinal axis 70 of distal tip 28, so that the threshold may be between about 8 mm and 10 mm.

Note that in response to movement of distal tip 28 away from SVs 65 and 66, processor 77 is configured to alter the transparency of SVs 65 and 66 again, such that tissue 44 is opaque, and thereby, obstructs the visibility of the inner volume of heart 12 at SVs 65 and 66.

FIG. 3 is a flow chart that schematically illustrates a method for dynamically adjusting transparency of one or more sub-volumes in an EA map 60 of heart 12, in accordance with an example of the present disclosure.

The method begins at a tag position receiving step 100, with processor 77 receiving positions of tags 63 and 64 within (the map of) heart 12. It is noted that processor 77 typically produces anatomical map 20, but in other examples, processor 77 may receive the map stored in any suitable memory of system 10. Moreover, processor 77 may receive the threshold described in FIG. 2D above, which may be used as a decision threshold for altering transparency level of tissue 44 responsively to the distance between distal tip 28 and closest tag (s) 64.

At a catheter position tracking step 102, processor 77 receives from position sensor 29 and/or from the ACL system, position signals indicative of the position and orientation of distal tip 28 being moved by physician 24 within heart 12, as described in detail in FIGS. 1 and 2A-2D above.

At a decision step 104, processor 77 is configured to check whether the distance between distal tip 28 and the closest tag (or another sort of predefined annotation) is smaller than the threshold. As shown in the example of FIG. 2D, processor 77 checks whether distance 68 (between distal tip 28 and tag 64a) is larger or smaller than the threshold stored in processor 77.

At a transparency altering step 106, in response to identifying the distance 68 is smaller than the threshold, processor 77 is configured to dynamically alter the transparency level in sub-volume 65 or in any other sub-volume of map 60D that is surrounding distal tip 28 and at least closest tag 64a.

At a displaying step 108, based on the altered transparency level received from processor 77, display device 27 is configured to display to physician 24, the sub-volume surrounding both distal tip 28 and at least the closest tag (s), e.g., tag 64a. It is noted that in case physician 24 moves distal tip 28, the method may loop back to step 102, but in case physician 24 is not interested in visiting additional sites within heart 12, the method is concluded at step 108. In general terms, display device 27 is configured to display (i) the anatomical map (e.g., EA map 60D) using a first transparency level (e.g., opaque view of tissue 44), and (ii) the sub-volume (e.g., SV 66) surrounding distal tip 28 using a second transparency level (e.g., transparent view), which is different from the first transparency level. Note that in the examples shown in FIGS. 2A-2D, the sub-volumes of heart 12 are either fully transparent or fully opaque. In other examples, however, processor 77 may determine any suitable transparency level to every sub-volume of heart 12, the transparency level may be predefined, or based on the features of interest (e.g., tags 64) located within the sub-volumes of interest of heart 12.

With reference to step 104, in case distance 68 is larger than the stored threshold, the method proceeds to opaque view displaying step 110, in which processor 77 is configured to t set an opaque view in the sub-volume surrounding distal tip 28. In this example, the inner volume of heart 12, at the sub-volume surrounding distal tip 28, is obstructed by tissue 44 of heart 12, and the method loops back to step 102 while physician 24 moves distal tip 28 within the cavities (e.g., chambers) of heart 12. Note that in case physician 24 is not interested in visiting additional sites within heart 12, the method may be concluded at step 110.

In alternative examples of step 110, processor 77 and display device 27 are configured to display the inner volume of heart 12 (e.g., using a transparent view) at a predefined area surrounding distal tip 28. In one implementation shown in inset 69 of FIG. 2D above, processor 77 and display device 27 are configured to use the transparent view for displaying tags 63 and 64 within SV 66. In another implementation, processor 77 and display device 27 may use the transparent view for displaying, in heart 12, an inner volume surrounding distal tip 28, regardless of whether or not tags or other sort of annotations at located at the displayed inner volume.

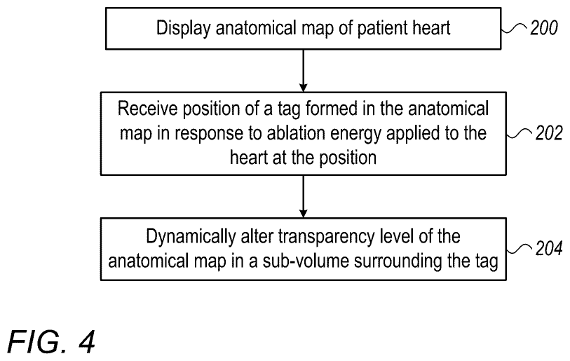

FIG. 4 is a flow chart that schematically illustrates a method for dynamically adjusting transparency of sub-volumes having ablation tags 63 and 64 in EA map 60 of heart 12, in accordance with another example of the present disclosure.

The method begins at an anatomical map displaying step 200 with processor 77 displaying anatomical map 20 or at least a portion of map 20, such as map 60A shown in FIG. 2A above.

At a tag receiving step 202, processor 77 receives the position of a new tag 64 formed in the anatomical map in response to ablation energy applied to heart 12 at the position of the tag. In some examples, processor 77 generates the tag after controlling the ablation process at the aforementioned position, and therefore, processor 77 may determine, rather than receive, the position of the new tag 64.

At a transparency altering step 204 that concludes the method, processor 77 is configured to dynamically alter the transparency level of the anatomical map in a sub-volume surrounding the new tag 64, as shown in the examples of maps 60B-60D shown in FIGS. 2B-2D above.

Note that in the method of FIG. 3 the transparency level of the selected sub-volume in the anatomical map depends on the position of distal tip 28. In the method of FIG. 4, however, the transparency level of the sub-volume surrounding the new tag 64 is altered regardless the position of distal tip 28.

In some examples, the anatomical map may have a plurality of tags, e.g., first and second tags formed after ablating tissue first and second locations, at respectively. In such examples, processor 77 is configured to dynamically alter the transparency level of the anatomical map: (i) in a first sub-volume surrounding the first tag, and subsequently, (ii) in a second sub-volume surrounding the second tag, while retaining the altered transparency level of the first sub-volume. For example, (i) physician 24 may perform the first ablation, the first tag is produced responsively, and processor 77 makes the outer surface of the heart transparent at the first sub-volume for displaying the first tag, subsequently, (ii) the second tag is produced responsively to the physician 24 performing the second ablation, and processor 77 (a) makes the outer surface of the heart transparent at the second sub-volume for displaying the second tag, and (b) retains the transparency of the first sub-volume for displaying the first tag. In other words, the altered transparency level of the first and second sub-volume is cumulative and follows the number of new tags formed after ablating the tissue at new respective locations during the ablation procedure.

### Example 1

A system (10) comprises a processor (77) and a display (27). The processor is configured to receive, in an anatomical map (20, 60A-60D) of an organ (12), a tracked position of a distal tip (28) of a catheter (14) that is moved within the organ, and (b) dynamically alter a transparency level in a sub-volume (62, 65, 66) of the anatomical map of the organ surrounding the distal tip responsively to the tracked position. The display is configured to display (i) the anatomical map using a first transparency level, and (ii) the sub-volume surrounding the distal tip using a second transparency level, different from the first transparency level.

### Example 2

The system according to Example 1, wherein the organ comprises a heart and the distal tip of the catheter comprises one or both of: (i) one or more sensing electrodes configured for sensing electro-anatomical signals when placed in contact with tissue of the heart, and (ii) one or more ablation electrodes configured to apply ablation energy when placed in contact with the tissue of the heart.

### Example 3

The system according to Example 2, wherein the anatomical map comprises at least a tag, which is displayed at a given location over the anatomical map and is indicative of an attribute of the heart at the given location.

### Example 4

The system according to Example 3, wherein the processor is configured to alter a size of the sub-volume responsively to a distance between the tracked position of the distal tip and a position of the tag.

### Example 5

The system according to Example 2, wherein the first transparency level comprises an opaque view of an outer surface of the anatomical map for visualizing to a user a three-dimensional (3D) topography of the anatomical map.

### Example 6

The system according to Example 2, wherein the second transparency level comprises a fully transparent view of an outer surface in the sub-volume of the anatomical map for displaying to a user a feature of the organ within the sub-volume surrounding the distal tip.

### Example 7

The system according to Example 1, wherein when the distal tip is moved and reaching a given position within the organ, the processor is configured to dynamically alter the transparency level within two seconds after the distal tip has reached the given position.

### Example 8

### A System (10), Comprising:

a processor (77), which is configured to receive: (i) a first position of a tag (64a) that is located within an inner volume of an anatomical map (60D) of an organ (12), and is indicative of an attribute of the organ at the first position, and (ii) one or more second positions of a distal tip (28) of a catheter (14) being moved within the organ, wherein, when a distance (68) between the first position and at least one of the second positions is smaller than a threshold, the processor is configured to alter a transparency level of a sub-volume of the anatomical map that contains the distal tip and the tag; and

a display (27), which is configured to display the sub-volume and the anatomical map to a user (24).

### Example 13

### A Method, Comprising:

receiving, in an anatomical map (60D) of an organ (12), a tracked position of a distal tip (28) of a catheter (14) that is moved within the organ;

dynamically altering a transparency level in a sub-volume (62) of the anatomical map of the organ surrounding the distal tip responsively to the tracked position; and

displaying (i) the anatomical map using a first transparency level, and (ii) the sub-volume surrounding the distal tip using a second transparency level, different from the first transparency level.

### Example 19

### A Method, Comprising:

receiving, in an anatomical map (60AC, 60D) of an organ (12), a position of a tag (63, 64) formed in the organ responsively to a medical procedure carried out at the position;

dynamically altering a transparency level in a sub-volume (62) of the anatomical map of the organ surrounding the tag; and

displaying (i) the anatomical map using a first transparency level, and (ii) the sub-volume surrounding the tag using a second transparency level, different from the first transparency level.

Although the examples described herein mainly address techniques for dynamically altering the transparency level of sub-volumes in patient heart during an electrophysiological (EP) procedure. The methods and systems described herein can also be used in other applications, such as in dynamically displaying inner volumes or surfaces in any other suitable organs of a patient.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A method, comprising:
receiving, in an anatomical map of an organ, a position of a tag formed in the organ responsively to a medical procedure carried out at the position;
dynamically altering a transparency level in a sub-volume of the anatomical map of the organ surrounding the tag; and
displaying (i) the anatomical map using a first transparency level, and (ii) the sub-volume surrounding the tag using a second transparency level, different from the first transparency level.

2. The method according to claim 1, wherein the organ comprises a heart, the medical procedure comprises an ablation energy applied to tissue of the heart at the position, and the tag is indicative of the ablation energy applied to the tissue.

3. A system, comprising:
a processor, which is configured to:
receive, in an anatomical map of an organ, a tracked position of a distal tip of a catheter that is moved within the organ and presented over the anatomical map; and
dynamically alter a transparency level in a sub-volume of the anatomical map of the organ surrounding the distal tip responsively to the tracked position; and
a display, which is configured to display (i) the anatomical map using a first transparency level, and (ii) the sub-volume surrounding the distal tip using a second transparency level, different from the first transparency level.

4. The system according to claim 3, wherein the organ comprises a heart and the distal tip of the catheter comprises one or both of: (i) one or more sensing electrodes configured for sensing electro-anatomical signals when placed in contact with tissue of the heart, and (ii) one or more ablation electrodes configured to apply ablation energy when placed in contact with the tissue of the heart.

5. The system according to claim 4, wherein the anatomical map comprises at least a tag, which is displayed at a given location over the anatomical map and is indicative of an attribute of the heart at the given location.

6. The system according to claim 5, wherein the processor is configured to alter a size of the sub-volume responsively to a distance between the tracked position of the distal tip and a position of the tag.

7. The system according to claim 4, wherein the first transparency level comprises an opaque view of an outer surface of the anatomical map for visualizing to a user a three-dimensional (3D) topography of the anatomical map.

8. The system according to claim 4, wherein the second transparency level comprises a fully transparent view of an outer surface in the sub-volume of the anatomical map for displaying to a user a feature of the organ within the sub-volume surrounding the distal tip.

9. The system according to claim 3, wherein when the distal tip is moved and reaching a given position within the organ, the processor is configured to dynamically alter the transparency level within two seconds after the distal tip has reached the given position.

10. A system, comprising:
a processor, which is configured to receive: (i) a first position of a tag that is located within an inner volume of an anatomical map of an organ, and is indicative of an attribute of the organ at the first position, and (ii) one or more second positions of a distal tip of a catheter being moved within the organ, wherein, when a distance between the first position and at least one of the second positions is smaller than a threshold, the processor is configured to alter a transparency level of a sub-volume of the anatomical map that contains the distal tip and the tag; and
a display, which is configured to display the sub-volume and the anatomical map to a user.

11. The system according to claim 10, wherein the processor is configured to estimate a movement direction of the distal tip based on the one or more second positions, and wherein the threshold comprises: (i) a first threshold along the movement direction, and (ii) a second threshold, different from the first threshold, along a given direction other than the movement direction.

12. The system according to claim 10, wherein the processor is configured to apply a first a transparency level to the anatomical map, and a second transparency level, which is different from the first transparency level to the sub-volume of the anatomical map that contains the distal tip and the tag.

13. The system according to claim 10, wherein the second transparency level is larger than the first transparency level for displaying to the user the sub-volume containing at least the distal tip and the tag.

14. The system according to claim 10, wherein the organ comprises a heart and the tag is based on at least one of an electrocardiogram signal and an intracardiac electrogram signal acquired when the distal tip is located at the first position.

15. A method, comprising:
receiving, in an anatomical map of an organ, a tracked position of a distal tip of a catheter that is moved within the organ;
dynamically altering a transparency level in a sub-volume of the anatomical map of the organ surrounding the distal tip responsively to the tracked position; and
displaying (i) the anatomical map using a first transparency level, and (ii) the sub-volume surrounding the distal tip using a second transparency level, different from the first transparency level.

16. The method according to claim 15, wherein the organ comprises a heart and the distal tip of the catheter comprises one or both of: (i) one or more sensing electrodes for sensing electro-anatomical signals when placed in contact with tissue of the heart, and (ii) one or more ablation electrodes for applying ablation energy when placed in contact with the tissue of the heart.

17. The method according to claim 16, wherein the anatomical map comprises at least a tag, which is displayed at a given location over the anatomical map and is indicative of an attribute of the heart at the given location.

18. The method according to claim 17, and comprising altering a size of the sub-volume responsively to a distance between the tracked position of the distal tip and a position of the tag.

19. The method according to claim 15, wherein displaying the anatomical map comprises displaying an opaque view of an outer surface of the anatomical map for visualizing to a a three-dimensional user (3D) topography of the anatomical map.

20. The method according to claim 15, wherein displaying the sub-volume surrounding the distal tip comprises displaying a fully transparent view of an outer surface in the sub-volume of the anatomical map for visualizing to a user a feature of the organ within the sub-volume surrounding the distal tip.
